(19) United States
(12) Patent Application Publication
(10) Pub. No.: US 2014/0018866 A1
(43) Pub. Date: Jan. 16, 2014

(54) SURGICAL SCREW ASSEMBLY WITH INCREASED ARTICULATION

(71) Applicants: VASKRSIJE JANKOVIC, KENOSHA, WI (US); MILAN D STEFANOVIC, SR., PLEASANT PRAIRIE, WI (US)

(72) Inventors: VASKRSIJE JANKOVIC, KENOSHA, WI (US); MILAN D STEFANOVIC, SR., PLEASANT PRAIRIE, WI (US)

(21) Appl. No.: 13/733,141

(22) Filed: Jan. 2, 2013

Related U.S. Application Data
(60) Provisional application No. 61/582,367, filed on Jan. 1, 2012.

(51) Int. Cl. A61B 17/86

(52) U.S. Cl. CPC A61B 17/8605
USPC 606/308

(57) ABSTRACT

A system, method, and apparatus for a surgical screw with increased articulation. In one embodiment, the apparatus includes a saddle; a split collet; a fastener with a head that fits inside the split collet; a cage for housing the saddle and split collet.

### CROSS-REFERENCE TO RELATED APPLICATIONS

This application claims priority to and benefit of co-pending U.S. provisional Patent Application No. 61/582,367, Attorney Docket Number VJ-102-PR1, filed on Jan. 1, 2012, entitled “SURGICAL SCREW WITH IMPROVED ARTICULATION,” by Vaskrsije Jankovic et al., which application is also incorporated by reference herein in its entirety.

### FIELD OF TECHNOLOGY

This disclosure relates generally to the technical fields of medical products, and in one example embodiment, this disclosure relates to a method, apparatus and system of surgical screws.

### BACKGROUND

Surgical screws are used for a wide variety of applications such as repairing broken or misaligned bones. A particular application is the repair of broken or misaligned vertebrae either from congenital defects or from traumatic injury. Fasteners are driven into a patient's bone for anchoring. The head of the fastener is then coupled to a supporting structure, e.g., a rod for a spinal column alignment procedure. Due to natural curvature of the human spine and vertebral elements, and sometimes due to the traumatic injury or congenital defects, such as scoliosis, the route of the supporting structure is not linear. Consequently, articulation is designed into a surgical screw and its mating elements, e.g., a retainer or receiver for the screw head.

### BRIEF DESCRIPTION OF THE VIEW OF DRAWINGS

Example embodiments are illustrated by way of example and not limitation in the figures of the accompanying drawings, in which like references indicate similar elements and in which:

FIGS. 1A-1C are orthogonal views of the surgical screw assembly including a top view, a cross-section view, and a bottom view, according to one or more embodiments.

FIGS. 2A-2B orthogonal views of the surgical screw assembly having a notched in the cage face to allow greater articulation, according to one or more embodiments.

FIG. 3 is an exploded view of the components of the surgical screw assembly, according to one or more embodiments.

FIGS. 4A-4B are orthogonal views including a top view and a cross-section view, respectively, of the clamshell used in the surgical screw assembly that allows greater articulation of the surgical screws, according to one or more embodiments.

FIGS. 5A-5B are isometric views of the fastener, and a close-up of the fastener head, respectively, according to one or more embodiments.

FIGS. 6A-6B are isometric views of the clamshell with a slot and the fastener with a rib, as alternative mating assembly features, according to one or more embodiments.

### DETAILED DESCRIPTION

A method, apparatus and system of a fastener assembly is disclosed. In the following description, for the purposes of explanation, numerous specific details are set forth in order to provide a thorough understanding of the various embodiments. It will be evident, however to one skilled in the art that various embodiments may be practiced without these specific details.

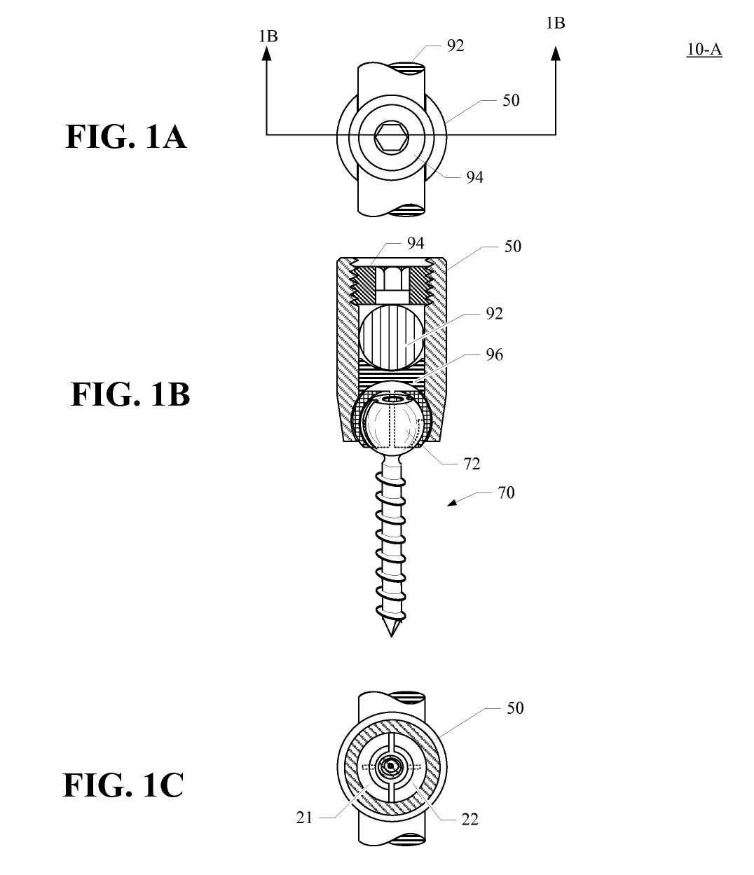

Referring now to FIGS. 1A-1C, orthogonal views of the fastener screw assembly including a top view, a cross-section view, and a bottom view are shown, according to one or more embodiments. Specifically, fastener assembly 10-A is shown in FIG. 1A as a top view, in FIG. 1B as a cross section, and FIG. 1C as a bottom view. The fastener assembly 10-A includes a cage 50 that houses and retains a retaining, or set, screw 94 that clamps down on a rod 92. The rod sits on a shaped saddle 96 for improved retainment of rod 92. Under saddle 96 sits a clamshell 20 and a fastener 70. The fastener assembly 10 provides adjustable articulation of the fastener 70 within clamshell 20 when retaining screw 94 is driven downward to compress rod 92 against saddle 96, wherein the saddle 96 in turn compresses and loads against a head 72 of fastener 70. The compressive clamping force of the saddle loads the clamshell assembly 20 and the head 72 of fastener 70 against a bottom end 54 of cage 50, and thus restricts movement of the fastener with respect to the cage 50. It is desirable to have as much articulation as possible between the fastener 70 and the cage 50, to accommodate a wider range of applications requiring said articulation. The clamshell assembly 20, also referred to as a collet, is made of multiple pieces, e.g., two pieces of a left half, or hemisphere, 21 and a right half, or hemisphere 22. A bottom surface of clamshell assembly 20 is slightly visible in FIG. 1C, in the circle smaller than the cross hatched face of cage 50. The fastener assembly 10 is utilized for a surgical application, but the present description is applicable to any use of a fastener.

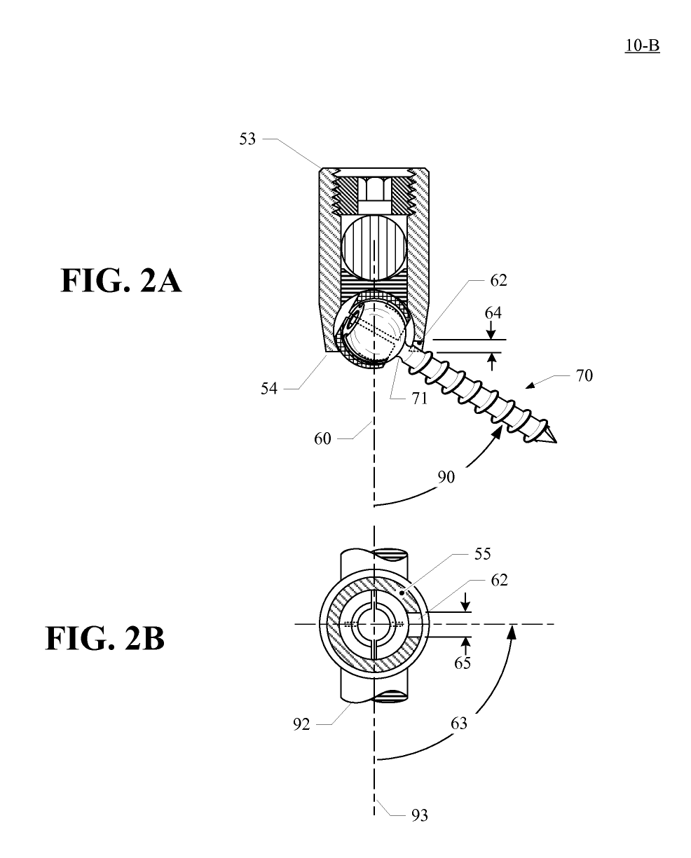

Referring now to FIGS. 2A-2B, orthogonal views of the surgical screw assembly having a notched in the cage face to allow greater articulation are shown, according to one or more embodiments. Cage 50 has a top end 53 and a bottom end 54, forming and axis 60 running between the ends, and between which the previously mentioned fastener assembly components are trapped. A notch 62, having a cutout height 64 and width 65, formed in face 55 of bottom end 54, at a radially clocked location 63 that is approximately 90 degrees in the present embodiment to the axis 93 of rod 92. The angular location of notch is to provide strength to cage 50 in a direction where the length of rod 92 cannot exert as much leveraged force on the assembly, e.g., the sideways leverage from the rod 92 on the assembly is not as great as the in-line leverage rod 92 can exert when the extended length of rod 92 can form a lever. A significantly improved articulation angle 90 is provided as fastener 70 is articulated with its neck 71 aligned with notch 62. The width 65 of notch 62 is approximately equal or greater than a neck diameter 72 of fastener 70, such that fastener can articulate without striking face 55 of second end 54 of cage 50. Notch 62 in one embodiment is a square cutout, while in another embodiment it is a circular cut to reduce stress concentration factors and to provide a cradle for neck 72 of fastener 70, thereby improving the integrity of the assembly and installation.

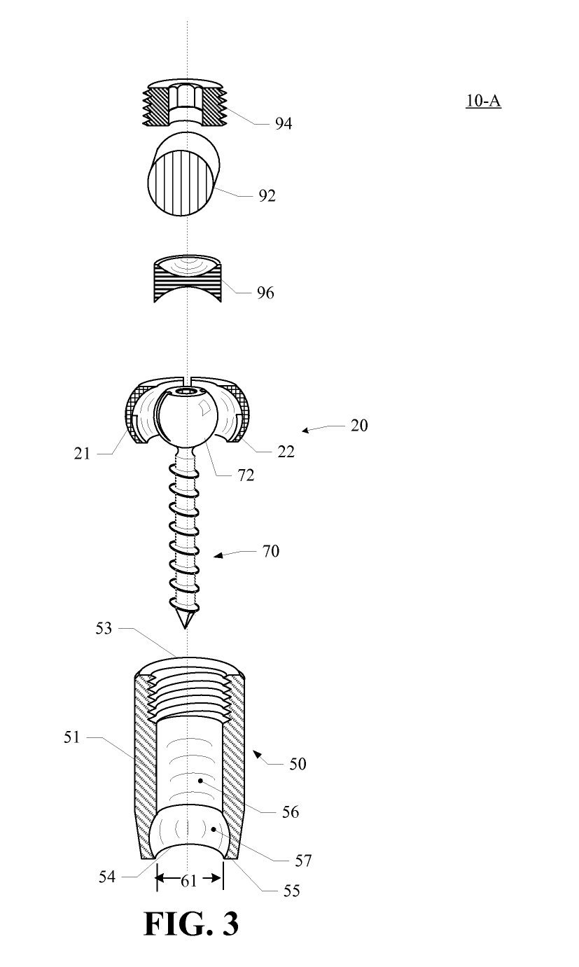

Referring now to FIG. 3, an exploded view of the components of the surgical screw assembly are shown, according to one or more embodiments. As shown, left half 21 and right halves 22 of clamshell assembly. An opening 61 at second end 54 of cage 50 allows fastener 70 to protrude from cage 50, but it is small enough to trap head 72, when enclosed by clamshell assembly 20.

Cage 50 has a body 51 with a cylindrical inner surface 56 through most of its body, with a threaded inside diameter at top end 53 to accommodate retainer screw 94. Bottom end of cage 50 has a spherical surface 57, with a radius from a center point, to accommodate the substantially spherical outside surface of clamshell assembly 20, and by virtue of being mated to each other, the substantially spherical inside surface of clamshell assembly 20 and finally the substantially spherical surface of head 72 of fastener. Substantially means that each of these surfaces is not a complete sphere, having protrusions, grooves, flat surfaces, truncated ends, and other discontinuities formed thereon or therein. However, the contact portions between the parts are substantially spherical, with an optional exception for the mated rib and groove, which perform an optional wedge action, described in a subsequent figure.

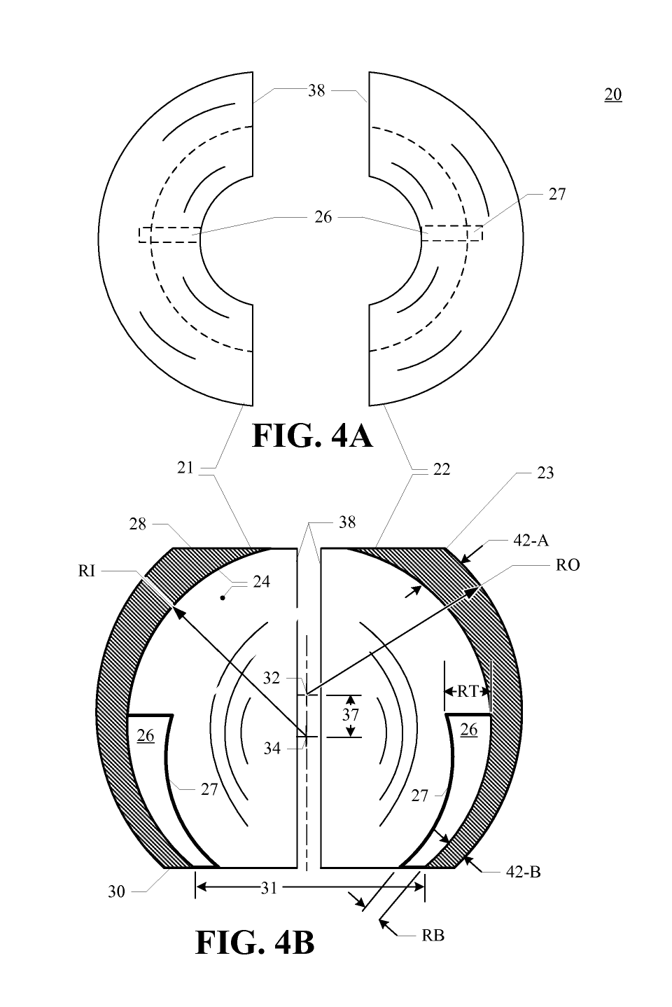

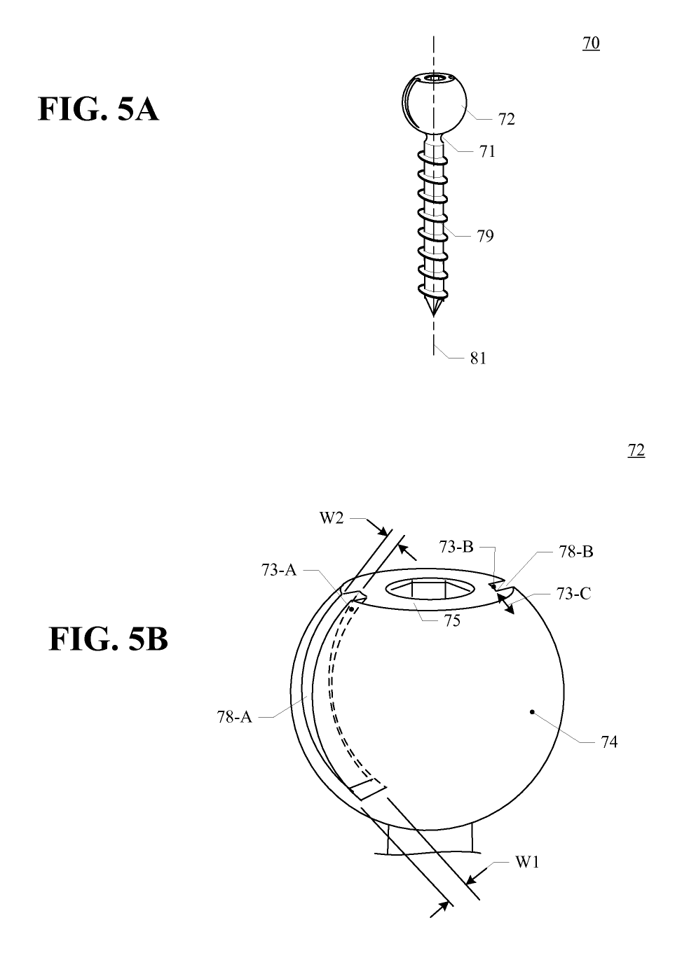

Referring now to FIGS. 4A-4B, orthogonal views including a top view and a cross-section view are shown, respectively, of the clamshell used in the surgical screw assembly that allows greater articulation of the surgical screws, according to one or more embodiments. Clamshell assembly 20 is comprised of multiple pieces, which are two halves 21, 22, in the present embodiment that mate together at approximately a split line 38 to form the spherical outer surface 23. The present embodiment splits the clamshell assembly along a vertical split line 38, which is in the same plane, e.g., coplanar, as axis of offset 37, as well as cage axis, or centerline, 60 of FIG. 2A, and other axes, such as an axis 81 of fastener 70, that runs along its body 79 as shown in FIG. 5A, when fastener 70 is axially aligned with axis 60 of cage 50.

Clamshell assembly 20, formed by its halves 21, 22, has an outer surface 23 and an inner surface 24 both of which are substantially spherical. In top end 28, which is flat, of clamshell assembly 20, is a top opening 31-A of sufficient diameter to enable access to hex wrench access to an hex drive fitting 76 in head 72 of fastener 70, shown in FIG. 5A In bottom end 30, which is flat, of clamshell assembly 20 is a bottom opening 31-B of sufficient diameter to enable access of body 79 of fastener 70 to pass there through, when assembled, as shown in FIGS. 1A-1C, and FIGS. 2A-2B. The center of the. openings 31-A, 31-B are coaxial with axis 81 of fastener 70 as the two retain their spherical orientation with each other, and the bottom opening 31-B must approximately align with body 81 of fastener 70.

Inside surface 24 is a portion of the clamshell assembly 20 that is in contact with the spherical head 72 of fastener 70. Inner surface 24 of the clamshell assembly 20-A is a substantially spherical inner surface having an inside radius (IR) from a center point 34. Outer surface 23 of the clamshell assembly 20-A is substantially spherical, having an outside a radius (OR) from a center point 32. The spherical surfaces allow articulation of both the head 72 of fastener 70 inside clamshell assembly 20-A, and the clamshell assembly 20-A within cage 50 the substantially spherical outer surface. The substantially spherical inner surface 24 of clamshell assembly 20-A is not concentric, but rather eccentric, with the substantially spherical outer spherical surface 23 of the clamshell assembly 20-A. The center point 34 of the inner spherical surface 24 is offset a distance 37 from the center point 32 outer spherical surface 23 of the clamshell assembly 20-A along an axis 36 formed by the noted centers. The offset allows the head 72 of fastener 70 to position itself lower in the fastener assembly 20-A, and thereby have more clearance from its neck 71 to the second end 54 of cage 50. Because of centers 32, 34 have offset 37, clamshell assembly 20-A resultantly has a cross-sectional thickness that varies, e.g., becomes thinner, from a top end 31-A to the bottom end 31-B of the clamshell 20-A, e.g., along the axis 36 for the openings in the clamshell and the axis of the offsets 32 and 34 of the outside surface 23 and 24, respectively.

Clamshell assembly 20-A has a mating feature that mates with the head of the fastener. The mating feature is a timed interlocking feature in one embodiment that is a rib 26 in each half 21, 22 of clamshell assembly 20-A. In particular, wedge surface 27 having a surface 27 that is flat in at one direction 25, but also curved, mates with slot 78-A and 78-B in head 72 of fastener 70, as shown in FIG. 5B. The wedge action pushes radially out on the clamshell assembly 20-A, perpendicular to axis 60, when seated in cage 50 and compressed from retaining screw 94, thus locking the assembly. The wedge action is important to prevent excessive loading on end 54 of cage 50, which is a weak area of the cage 50 body 51 because of the opening therein, and because it is sometimes fabricated from weaker material than fastener 70 and clamshell 20

Referring now to FIGS. 5A-5B, isometric views of the fastener, and a close-up of the fastener head are shown, respectively, according to one or more embodiments. Fastener 70 includes a head 72, a neck 71, and a threaded body 79. Head 72 has an outer surface 74 that is substantially spherical, for seating against the clamshell assembly 20-A.

Outer surface 74 of head 72 the outer surface of the head of the fastener has a mating interlocking feature with the clamshell, as shown in FIG. 5B, that is a slot surface 78-A, 78-B disposed at opposite sides of head 74, e.g., 180 degrees, that have a substantially flat surface 73-B in at least one direction 73-C to mate with surface 27 of clamshell assembly 20-A. Depth of slot 78-A, 78-B varies from top surface 75 of head 72 to bottom of head 72 by neck 71. This is create more wedge action in one embodiment, though in another embodiment only the fastener 70 or the clamshell assembly 20-A has a wedge shape surface. Likewise, width W2 of slot 78-A, 78-B can be constant from top to bottom, but in another embodiment it narrows on either the top or bottom.

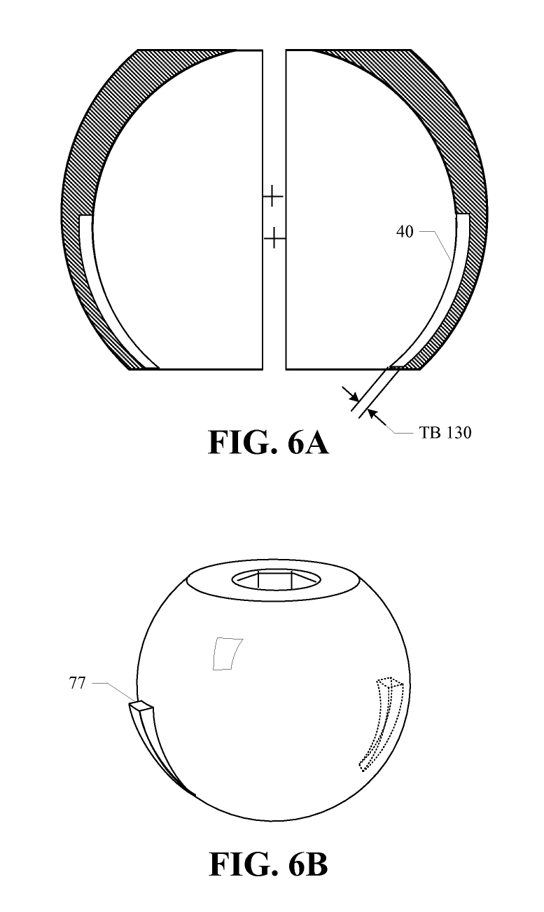

Referring now to FIGS. 6A-6B, isometric views of the clamshell with a slot and the fastener with a rib are shown, respectively, as alternative mating assembly features, according to one or more embodiments. Clamshell assembly 20-A has a slot 40 rather than a protruding rib at a depth of TS, while fastener head has a protruding rib 77 with a wedge effect, as it approaches the center height of head 72.

While the present embodiment utilizes two pieces to form the substantially spherical body, the clamshell can be any number of pieces in another embodiment. Also, while the present embodiment provides a vertical split line, another embodiment can provide a horizontal split line, with the bottom half having an opening for body 79 of fastener 70, and with a single cut in the circumference to allow expansion and loading of the inside surface of cage 50 for reduced loading on second end of cage and hence improved structural integrity of fastener assembly. In another embodiment, the thickness of the clamshell is thicker at the bottom to offer more strength. In addition, rib configuration ion another embodiment uses a point edge, rather than a flat surface, or uses detents to push radially outward.

### ALTERNATIVES

References to methods, operations, processes, systems, and apparatuses disclosed herein that are implementable in any means for achieving various aspects, and may be executed in a form of functions or operations may include retaining, trapping, articulating, aligning, rotating, seating, and the like.

Methods and operations described herein can be in different sequences than the exemplary ones described herein, e.g., in a different order. Thus, one or more additional new operations may be inserted within the existing operations or one or more operations may be abbreviated or eliminated, according to a given application, so long as substantially the same function, way and result is obtained.

Although the present embodiments have been described with reference to specific example embodiments, it will be evident that various modifications and changes may be made to these embodiments without departing from the broader spirit and scope of the various embodiments.

The foregoing descriptions of specific embodiments of the present disclosure have been presented for purposes of illustration and description. They are not intended to be exhaustive or to limit the invention to the precise forms disclosed. Many modifications and variations are possible in light of the above teaching without departing from the broader spirit and scope of the various embodiments. The embodiments were chosen and described in order to explain the principles of the invention and its practical application the best, to enable others skilled in the art to best utilize the invention and various embodiments with various modifications as are suited to the particular use contemplated. It is intended that the scope of the invention be defined by the Claims appended hereto and their equivalents.

I/we claim:

1. A fastener assembly having selectable articulation, the device comprising:
a clamshell for retaining a head of a fastener;
a cage for retaining the clamshell; and
wherein:
the clamshell has an outer surface and an inner surface;
the outer surface of the clamshell is substantially spherical to allow articulation of the clamshell within the cage, the substantially spherical outer surface having a radius from a center point; and
the clamshell has an inner surface with a mating feature that mates with the head of the fastener.

2. The assembly of claim 1 wherein:
the mating feature on the inner surface of the clamshell is a wedge surface, the wedge surface for mating against the head of the fastener.

3. The assembly of claim 2 wherein:
the wedge surface of the clamshell is a flat surface in at least one direction that mates with a surface on the head of the fastener;
the surface on the head of the fastener is flat in one direction.

4. The assembly of claim 3 wherein:
the wedge surface is a rib that extends out from, or a slot that extends into, the inner surface of the clamshell.

5. The assembly of claim 2 further comprising:
a fastener having a head;
wherein:
the head has an outer surface that is substantially spherical, the outer surface of the head for seating against the wedge of the clamshell to seat the head in the cage.

6. The assembly of claim 5 wherein:
the outer surface of the head of the fastener has a mating interlocking feature with the clamshell.

7. The assembly of claim 1 wherein:
the inner surface of the clamshell is a substantially spherical inner surface having a radius from a center point; and
the substantially spherical surface of the inner surface of the clamshell is not concentric with the substantially spherical outer spherical of the clamshell.

8. The assembly of claim 1 wherein:
the inner spherical surface of the clamshell is offset from the outer spherical surface of the clamshell along an axis formed by the center of the inner spherical surface and by the center of the outer spherical surface.

9. The assembly of claim 8 wherein:
the cage has a body with a first end that has an opening and a second end that has an opening and has an axis extending along the body from the first end to the second end; and
the axis of the cage is parallel to the axis of the offset for the clamshell.

10. The assembly of claim 8 wherein:
the second end of the cage has a notched surface extending from the opening;
the width of the notch is approximately equal to a diameter of a neck of the fastener; the notched surface for allowing an extended articulation of the fastener with respect to the cage.

11. The assembly of claim 8 wherein:
the cage has an inside surface that includes a cylindrical surface at the first end and that includes a spherical surface at the second end, the spherical surface of the cage having a radius from a center point;
the spherical surface of the inside surface of the cage for housing the spherical outside surface of the clamshell; and
the opening has a diameter for passing a body of the fastener there through.

12. The assembly of claim 4 wherein the clamshell comprises:
a plurality of pieces having split lines that mate together to form the spherical outer surface of the clamshell; and
wherein the split lines of the plurality of pieces are coplanar with an axis of a fastener to which it retains in the assembly.

13. The assembly of claim 1 wherein:
the fastener articulates with respect to the cage when the fastener is not seated; and
the fastener does not articulate with respect to the cage when the fastener is seated.

14. The assembly of claim 5 wherein:
the wedge surface of the clamshell exerts a force against the inner surface of the cage in a radial direction when a load is exerted to seat the fastener inside the cage.

15. The assembly of claim 5 wherein:
the clamshell has a first opening and a second opening that form an axis for the openings;
the axis for the openings of the clamshell are coaxial with the offset axis of the clamshell; and
a portion of the clamshell in contact with the spherical head of the fastener has a cross-sectional thickness that varies from a first end of the clamshell to a second end of the clamshell along the axis for the openings in the clamshell.

16. A fastener assembly having selectable articulation, the device comprising:
a clamshell for retaining a head of a fastener;
a cage for retaining the clamshell; and
wherein:
the clamshell is a multi-piece clamshell that floats within the cage;
the clamshell has a first opening and a second opening that form an axis for the openings;
the axis for the openings of the clamshell are coaxial with the offset axis of the clamshell; and
a portion of the clamshell in contact with the spherical head of the fastener has a cross-sectional thickness that varies from a first end of the clamshell to a second end of the clamshell along the axis for the openings in the clamshell.

17. The assembly of claim 16 further comprising:
a fastener having a head, wherein the head has an outer surface, and the outer surface of the head seats against the wedge of the clamshell to seat the head in the cage; and
wherein:
the clamshell has an outer surface and an inner surface;
the outer surface of the clamshell is spherical to allow articulation of the clamshell within the cage, the spherical surface having a radius from a center point;
the clamshell has an inner surface with an mating feature that mates with the head of the fastener; and
the mating feature on the inner surface of the clamshell is a wedge surface, the wedge surface for mating against the head of the fastener.

18. The assembly of claim 16 wherein:
the clamshell has an outer surface and an inner surface;
the outer surface of the clamshell is spherical to allow articulation of the clamshell within the cage, the spherical surface having a radius from a center point; and
the clamshell has an inner, surface with a mating feature with the head of the fastener.

19. A fastener assembly having selectable articulation, the device comprising:
a fastener having a head;
a clamshell for retaining the head of the fastener; and
a cage for retaining the clamshell, wherein:
the cage has a body with a first end, that has an opening and a second end;
the head of the fastener is trapped in the cage;
the fastener can articulate with respect to the cage; and
the cage has a notch cut into the first end to allow the fastener to articulate.
